(19) United States
(12) Patent Application Publication
(10) Pub. No.: US 2004/0122048 A1
(43) Pub. Date: Jun. 24, 2004

(54) Stabilized pharmaceutical composition containing basic excipients

(75) Inventors: Eric Joel Benjamin, Pomona, NY (US); Maysara Saleh Rabah, Congers, NY (US)

Correspondence Address: WYETH, PATENT LAW GROUP, FIVE GIRALDA FARMS, MADISON, NJ, 07940, US

(73) Assignee: Wyeth Holdings Corporation, Madison, NJ

(21) Appl. No.: 10/675,161

(22) Filed: Sep. 30, 2003

Related U.S. Application Data
(60) Provisional application No. 60/417,804, filed on Oct. 11, 2002.

(51) Int. Cl. A61K 31/47

(52) U.S. Cl. 514/312; 514/313

(57) ABSTRACT

This invention provides a stable stabilized pharmaceutical composition in an oral dosage form comprising a compound of the formula

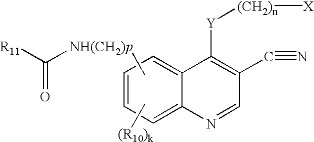

wherein:

R₁₁, R₁₀, R₅, R₆, R₇, R₈, R, Y, X, k, p and n are as defined herein, or its pharmaceutically acceptable salts, esters or ethers thereof.

This application claims priority from copending provisional application, Application No. 60/417,804 filed Oct. 11, 2002, the entire disclosure of which is hereby incorporated by reference.

### BACKGROUND TO THE INVENTION

This invention provides a stabilized pharmaceutical composition in an oral dosage form. Methods for making tablets and other oral dosage forms are well known. For example in Handbook of Pharmaceutical Granulation Technology, 1997, Dilip Parikh, Marcel Dekker, Inc. ISBN 0-8247-9882-1 and Pharmaceutical dosage forms: Tablets, Second Edition, Herbert Lieberman, Leon Lachman, and Joseph Schwartz, Marcel Dekker, Inc. ISBN 0-8247-8044-2, methods of making tablets and other oral dosage forms are described in detail.

Briefly, “dry blend” materials are physically blended together before filling capsules or compressing tablets. See, Handbook of Pharmaceutical Granulation Technology, 1997, Dilip Parikh, Marcel Dekker, Inc. ISBN 0-8247-9882-1, page 309.

In dry granulation (slugging or roller compaction) intragranular materials are blended to prepare slugs or roller compaction. Material is milled and blended with extragranular materials followed by capsule filling or tablet compression.

Wet granulation entails blending intragranular materials. Wet granulate the blend with water (using high sheer, low sheer granulators) and dry (using temeratures up to 100° C.). Material is milled and blended with extragranular materials followed by capsule filling or tablet compression. See, Handbook of Pharmaceutical Granulation Technology, 1997, Dilip Parikh, Marcel Dekker, Inc. ISBN 0-8247-9882-1, pages 338-368.

Excipients could be added as dry material to the blends or could be dissolved in the granulation fluid.

Wet granulation can also be done using fluid bed granulation, which combines granulation and drying steps above.

Extrusion/Spheronization is utilized in the preparation of spheres or beads.

The compounds described herein have been shown to inhibit Epidermal Growth Factor Receptor (EGF-R) kinase. Epidermal Growth Factor Receptor (EGF-R) kinase is a protein that contributes to tumor cell growth in the laboratory and with poor prognosis in tumor types in humans.

The compounds were previously claimed in U.S. Pat. No. 6,002,008 issued Dec. 14, 1999.

In U.S. Pat. No. 5,879,708, issued Mar. 9, 1999, a stable composition of benzenimidazole compounds were prepared using a basic inorganic salt (pH of not less than 7 when in the form of a 1% aqueous solution or suspension).

In EP 0 475 482 A1 and EP 0 475 482 B1 published Mar. 18, 1992 and Sep. 13, 2000 respectively, stabilization of a water-soluble acid addition salts of poorly soluble basic compounds (apomorphine, chlopromazine, imipramine, promethazine, and mainserin) in pharmaceutical compositions by addition of a water-soluble alkaline stabilizer are described.

In U.S. Pat. No. 4,743,450, issued May 10, 1998, the cyclization, hydrolysis, and coloration of certain ACE inhibitors (quinapril, inolapril or their pharmaceutically acceptable addition salts) is shown to be minimized when formulated with a metal-containing stabilizer and a saccharide.

In U.S. Pat. No. 4,382,091, issued May 3, 1983 the pharmaceutically acceptable acid addition salts of sulfur containing antimicrobial 1-substituted imidazole compounds are stabilized in talc based powders by addition of a basic metal salt of an inorganic or organic acid.

Zhu et al. “Stabilization of proteins encapsulated in cylindrical poly(lactide-co-glycolide) implants: mechanism of stabilization by basic excipients” Pharm. Res., 17:351-357, 2000 reports that the aggregation of albumin encapsulated in cylindrical poly(lactide-co-glycolide) implants was stabilized by incorporation of basic excipients such as magnesium hydroxide.

In Cotton et al. “L-649,923—The selection of an appropriate salt form and preparation of a stable oral formulation”, Int. J. Pharm., 109:237-249, 1994. L-649,923 the salt of γ-hydroxy acid in the oral dosage form having compound primarily in a solid state was stabilized by decreasing the amount of free acid in the drug substance, avoiding aqueous granulation process, and using excipients with low water content and adding sodium carbonate as a basic excipient.

### BRIEF SUMMARY OF THE INVENTION

This invention provides a stabilized pharmaceutical composition comprising a compound of the formula:

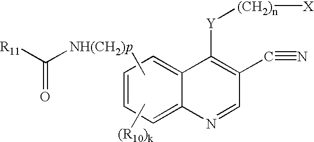

wherein:

X is selected from the group consisting of cycloalkyl or phenyl optionally substituted with one or more substituents selected from the group consisting of hydrogen, halogeno, alkyl of 1-6 carbon atoms, alkenyl of 2-6 carbon atoms, alkynyl of 2-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; the moieties (R₁₀)ₖ represent 1 to 3 substituents on the aromatic ring that can be the same or different and are selected independently from the group hydrogen, halogeno, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, halomethyl, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, trifluoromethyl, cyano, nitro, carboxy, carboalkyl of 2-7 carbon atoms, phenoxy, phenyl, thiophenoxy, benzyl, alkoxyamino of 1-4 carbon atoms, dialkylamino of 2 to 12 carbon atom, N,N-dialkylaminoalkyl of 3-14 carbon atoms, phenylamino, benzylamino, N-alkylcarbamoyl of 1-6 carbon atoms, N,N-dialkylcarbamoyl of 2-12 carbon atoms; R₁₁ is a radical and is selected from the group:

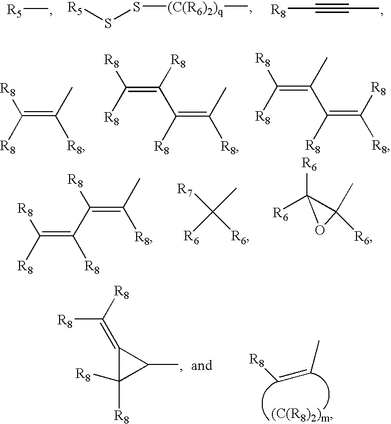

n is 0-1;

Y is —NH—, —O—, —S—, or —NR—;

R is alkyl of 1-6 carbon atoms;

R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;

R₆ is hydrogen, alkyl of 1-6 carbon atoms, or alkenyl of 2-6 carbon atoms;

R₇ is chloro or bromo;

R₈ is hydrogen, alkyl of 1-6 carbon atoms, aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;

k=1-3, q=1-3, m=1-3, and p=0-3; or a pharmaceutically acceptable salt thereof;

said pharmaceutical composition containing at least one basic excipient in a concentration sufficient to bring the pH of the composition to at least 8, and at least one pharmaceutically acceptable excipient.

The pharmaceutically acceptable salts are those derived from such organic and inorganic acids as: acetic, lactic, citric, tartaric, succinic, maleic, malonic, gluconic, hydrochloric, hydrobromic, phosphoric, nitric, sulfuric, methanesulfonic, and similarly known acceptable acids.

This invention also provides a stabilized pharmaceutical composition comprising a compound of the formula I as defined herein, at least one basic excipient and at least one pharmaceutically acceptable excipient; said basic excipient(s) being in an amount sufficient to stabilize the pharmaceutical composition.

This invention further provides a stabilized pharmaceutical composition comprising a compound of the formula I as defined herein, at least one basic excipient and at least one pharmaceutically acceptable excipient; said basic excipient(s) being in an amount from about 0.1% to about 50% by weight; preferably about 0.25% to about 10% by weight; most preferably from about 0.5% to about 5% by weight, of the pharmaceutical composition.

In a prefered embodiment of the invention the stabilized pharmaceutical comprises a compound of the formula:

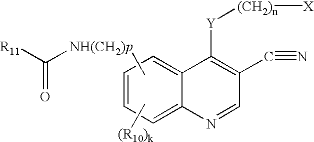

wherein:

X is a phenyl optioanlly substituted with a halogen;

n is 0-1;

Y is NH;

(R₁₀)_{K} is hydrogen, methoxy, ethoxy;

k=1-3, and p=0-3;

R₁₁ is

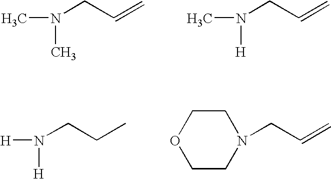

or a pharmaceutically acceptable salt thereof.

In the most prefered embodiment of the invention the compound comprises 4-dimethylamino-but-2-enoic acid [4-(3-chloro-4-fluoro-phenylamino)-3-cyano-7-ethoxy-quinolin-6-yl]-amide (EKB-569).

The alkyl portion of the alkyl or groups containing alkyl such as alkoxy, alkoxymethyl, alkanoyloxymethyl, alkylsulphinyl, alkylsulphonyl, alkylthio, carboalkoxy, carboalkyl, alkanoylamino, aminoalkyl, alkylaminoalkyl, N,N-dicycloalkylaminoalkyl, hydroxyalkyl, and alkoxyalkyl substituents include both straight chain as well as branched carbon chains. The cycloalkyl portions of N-cycloalkyl-N-alkylaminoalkyl and N,N-dicycloalkylaminoalkyl substituents include both simple carbocycles as well as carbocycles containing alkyl substituents. The alkenyl portion of the alkenyl or groups containing alkenyl such as alkenoyloxymethyl, alkenyloxy, alkenylsulfonamido, substituents include both straight chain as well as branched carbon chains and one or more sites of unsaturation. The alkynyl portion of the alkynyl or groups containing alkynyl such as alkynoyloxymethyl, alkynylsulfonamido, alkynyloxy, substituents include both straight chain as well as branched carbon chains and one or more sites of unsaturation. Carboxy is defined as a —CO₂H radical. Carboalkoxy of 2-7 carbon atoms is defined as a —CO₂R″ radical, where R″ is an alkyl radical of 1-6 carbon atoms. Carboalkyl is defined as a —COR″ radical, where R″ is an alkyl radical of 1-6 carbon atoms. Alkanoyloxy is defined as a —OCOR″ radical, where R″ is an alkyl radical of 1-6 carbon atoms. Alkanoyloxymethyl is defined as R″CO₂CH₂— radical, where R″ is an alkyl radical of 1-6 carbon atoms. Alkoxymethyl is defined as R″OCH₂— radical, where R″ is an alkyl radical of 1-6 carbon atoms. Alkylsulphinyl is defined as R″SO— radical, where R″ is an alkyl radical of 1-6 carbon atoms. Alkylsulphonyl is defined as R″SO₂— radical, where R″ is an alkyl radical of 1-6 carbon atoms. Alkylsulfonamido, alkenylsulfonamido, alkynylsulfonamido are defined as R″SO₂NH— radical, where R″ is an alkyl radical of 1-6 carbon atoms, an alkenyl radical of 2-6 carbon atoms, or an alkynyl radical of 2-6 carbon atoms, respectively. N-alkylcarbamoyl is defined as R″NHCO— radical, where R″ is an alkyl radical of 1-6 carbon atoms. N,N-dialkylcarbamoyl is defined as R″ R′NCO— radical, where R″ is an alkyl radical of 1-6 carbon atoms, R′ is an alkyl radical of 1-6 carbon atoms and R′, and R″ may be the same or different. When X is substituted, it is preferred that it is mono-, di-, or tri-substituted, with monosubstituted being most preferred. An azacycloalkyl-N-alkyl substituent refers to a monocyclic heterocycle that contains a nitrogen atom on which is substituted a straight or branched chain alkyl radical. A morpholino-N-alkyl substituent is a morpholine ring substituted on the nitrogen atom with a straight or branch chain alkyl radical. A piperidino-N-alkyl substituent is a piperidine ring substituted on one of the nitrogen atoms with a straight or branch chain alkyl radical. A N-alkyl-piperidino-N-alkyl substituent is a piperidine ring substituted on one of the nitrogen atoms with a straight or branched chain alkyl group and on the other nitrogen atom with a straight or branch chain alkyl radical. Halogen of this invention is a bromo, fluoro, or chloro group.

The compounds of this invention may contain an asymmetric carbon; in such case, the compounds of this invention cover the racemate and the individual R and S entantiomers, and in the case were more than one asymmetric carbon exists, the individual diasteromers, their racemates and individual entantiomers.

The pH of a stabilized composition of the invention may be assessed by suspending or dissolving 60-250 mg of the composition (if appropriate after crushing to form a powder) per 2 ml of aqueous medium, e.g. water.

For purposes of this invention a compound is considered to be stabilized when there is a decrease in the rate of degradation, loss of concentration, or physical change of the compound when compared to a reference compound without excipient. A compound can be judged to be stabilized when the rate of decrease in dosage form strength is minimized.

For purposes of this invention a basic excipient comprises basic inorganic salts, basic organic salts and basic organic compounds including, but not limited to, for example sodium carbonate, sodium bicarbonate, calcium carbonate, arginine, tromethamine and EDTA, sodium carbonate monohydrate, ammonium carbonate, glycine, and magnesium carbonate. The basic excipient is found in the pharmaceutical composition of this invention in a concentration that will bring the pH of the composition to at least 8. The basic excipient may be incorporated into the pharmaceutical composition either individually or in combination.

For purposes of this invention, a pharmaceutical acceptable excipient is a nonactive ingredient added to the tablet formulation. Excipients include, but are not limited to diluents, disintegrants; glidants; binder; lubricants; antioxidants; preservatives; coloring and flavoring agents; emulsifying and suspending agents; and pharmaceutical solvents. Osol et al., Remington's Pharmaceutical Sciences (16^{th} edition), 1980, 1225-1267and 1367 and Liberman, et al., Phamaceutical Dosage Forms: Tablets (volume 1), 1989, ISBN: 0-8247-8044-2, both of which are hereby incorporated by reference.

For purposes of this invention, filler is any compound added to the pharmaceutical composition to increase bulk, weight, viscosity, opacity, or strength. Examples of fillers include, but are not limited to, microcrystalline cellulose, avicel, and lactose. In an embodiment of this invention the microcrystalline cellulose and lactose may be found alone or in combination in the pharmaceutical composition.

In an embodiment of this invention, disintegrants apply to compounds added to the pharmaceutical composition for the purpose of causing the compressed composition (tablet) to break apart when placed in an aqueous environment. Examples of a disintegrant include, but are not limited to microcrystalline cellulose, avicel, and starch glycolate found alone or in combination in the pharmaceutical composition. Lieberman et al, (Id. at pages 108-110 and 173-177).

For purposes of this invention, glidants improve flow characteristics of the pharmaceutical composition and include talc, magnesium stearate, or silicon alone or in combination. (Id. at page 115-116 and 177-179).

For purposes of this invention, a binder is a material that holds the powders together to form granules. Examples of a binder include but are not limited to povidine and magnesium stearate. (Id. at page 105-108 and 160-168).

In an embodiment of this invention the pH of the stabilized pharmaceutical composition after addition of the basic excipient is from about 8 to about 13.5. In another embodiment the pH of the composition after addition of the basic excipient is from about 8 to about 10. In the most preferred embodiment the composition after addition of the basic excipient is 8.

In an embodiment of this invention the basic excipient combined with a pharmaceutically acceptable excipient alone or in combination has a concentration of about 0.1% to about 50% by weight of the pharmaceutical composition. In a preferred embodiment the concentration may be about 0.25% to about 10% by weight of the pharmaceutical composition. In the most preferred embodiment of this invention the concentration is about 0.5% to about 5% by weight of the pharmaceutical composition.

In an embodiment of this invention the stabilized pharmaceutical composition is a dosage form having compound primarily in a solid state. In another embodiment the pharmaceutical composition may be in a semi-solid form. In another embodiment the pharmaceutical composition may be in a suspension form. The pharmaceutical composition may be in an immediate release form.

For purposes of this invention, solid dosage form is a dosage form in which the compound is primarily present in a solid state and may be, for example, a powder, a sphere, a capsule, or a tablet.

For purposes of this invention a semi-solid form can be an ointment for external application to the body. An ointment should have the characteristics of compatibility with the skin, inertness, and ablity to release incorporated medication.

The solid dosage form can be enteric-coated, sugar coated, or film coated. See, Lieberman et al., Pharmaceutical Dosage Forms: Tablets (volume 3), 1990, ISBN: 0-8247-8300-X, pages 77-158, hereby incorporated by reference.

The following experimental details are set forth to aid in an understanding of the invention, and are not intended, and should not be construed, to limit in any way the invention set forth in the claims that follow thereafter.

### BRIEF DESCRIPTION OF THE DRAWINGS

FIG. 1: Degradation of crushed EKB-569 tablet slurry in water at various pH conditions. A plot of largest single impurity (LSI) versus pH.

FIG. 2: Levels of largest single impurity (LSI) for EKB-569 in crushed EKB-569 tablet slurries prepared using small quantities of 5% solutions or suspensions of basic materials. Samples were stored at 56° C.

FIG. 3: Levels of largest single impurity (LSI) for EKB-569 in crushed EKB-569 tablet slurries prepared using small quantities of 1% solutions or suspensions of basic materials (EDTA: 0.1% solution). Samples were stored at 56° C.

FIG. 4: Comparison between stability of EKB-569 25 mg capsule pharmaceutical compositions with and without 1% sodium carbonate. A plot of the change in level of impurity MWT440 versus Time. Samples stored at 40° C./75% RH. Similar tablet and capsule pharmaceutical compositions containing 1% sodium carbonate exhibit a similar stability profile. Capsule B contains 1% sodium carbonate. Capsule A contains no sodium carbonate.

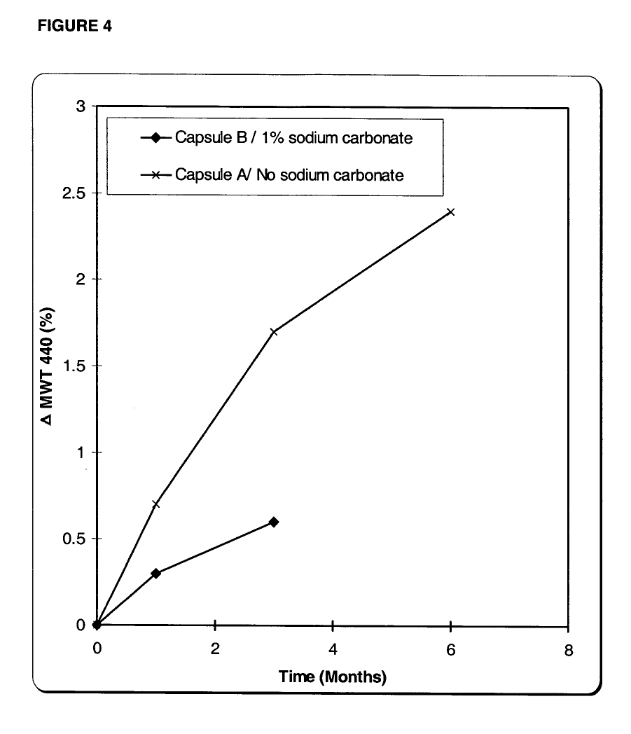

### DETAILED DESCRIPTION OF THE INVENTION

The compounds of this invention may be classified as BCS I compounds (soluble and permeable) based on the Biopharmaceutical Classification System, Amidone, G. L. et al, Pharm. Res. 12(3):413-420, 1995. The aqueous solubility of the compounds is dependent on pH; the compounds are soluble at low pH conditions, solubility decreases significantly between pH 4 and 6. The compounds are insoluble at pH values higher than 6.

The compounds demonstrate poor stability in the presence of water, heat and light. Table 1 shows the stability of EKB-569 in solution in the pH range of 1.2-9 at 56° C. and 80° C. The data indicates that EKB-569 is more stable in acidic solution and exhibits faster degradation in neutral and basic solutions.

TABLE 1
Stability of EKB-569 solution (0.2 mg/mL EKB-569
in 0.02 M buffer with 50% Acetonitrile) at 56° C.

EKB-569 degradation
rate constant
(/hour)

| Buffer solution | 56° C. | 80° C. |
|---|---|---|
| pH 1.2 (HCl) | — | 0.0013 |
| pH 3.0 (Phoshphate) | — | 0.0003 |
| pH 5.0 (Acetate) | 0.0008 | 0.0113 |
| pH 7.4 (Phosphate) | 0.003 | 0.0235 |
| pH 9 (Glycine) | 0.0031 | 0.0229 |

EKB-569 also exhibited chemical instability in the solid state. A study was conducted at 56° C./75% RH for 2 weeks. The samples were filled into 2-ml flame sealed Kimble score-break ampules. Results are shown in Table 2.

TABLE 2
Solid state stability for EKB-569 drug
substance at 56° C./75% RH:

| Storage period | % EKB-569 remaining | Total impurities |
|---|---|---|
| Initial | 99.1 | 0.78 |
| 3 Days | 98.1 | 1.1 |
| 7 Days | 97.7 | 1.68 |
| 14 Days | 97.1 | 2.62 |

Mechanism of degradation is not well-established. Degradation is mainly due to the cyclization of the dimethylamino-but-2-enoic acid side chain. The resulting compound has a molecular weight of 440 and considered the largest single impurity (LSI). Change in total impurities (TI) over storage is mainly due to the change in the level of this impurity.

The instability of solid drug substance complicated the handling of solid dosage forms for these compounds. A pharmaceutical composition of EKB-569 tablet is given in Table 3. This tablet exhibited poor stability at 40° C./75% RH (Table 4). This suggested that the EKB-569 tablet would require refrigeration to obtain acceptable shelf life and maintain effectiveness.

TABLE 3
Composition of EKB-569 tablet

| Compound + excipients | Formula 1% |
|---|---|
| EKB-569 | 7.143 |
| Avicel PH101 | 47.537 |
| Lactose, anhydrous | 40.0 |
| Sodium starch glycolate | 5.0 |
| Magnesium stearate | 0.5 |

TABLE 4
Stability data for EKB-569 tablet at 40° C./75% RH.

| | Largest single impurity | Total impurities |
|---|---|---|
| Time point | LSI % | TI % |
| Initial | 0.63 | 1.03 |
| 1 Months | 3.57 | 4.72 |
| 2 Months | 5.09 | 6.55 |

The present invention provides for stabilized orally administered pharmaceutical compositions for the exemplified compounds. The reactivity of the drug and its tendency to undergo degradation in the solid state is reduced by the addition of basic excipients that can bring the pH of pharmaceutical composition to 8 or above. Basic excipients include basic inorganic salts, organic salts, and organic compounds.

Results of studies conducted previously indicated that the pharmaceutical compositions were most stable at acidic conditions. Results also showed that degradation was highest at basic conditions (Table 1). The pH of a slurry of crushed EKB-569 tablet (Table 3) in about 3 ml of water was found to be 7.85. The data in Table 3 supported testing improved stability of a pharmaceutical composition by lowering the pH of the pharmaceutical composition.

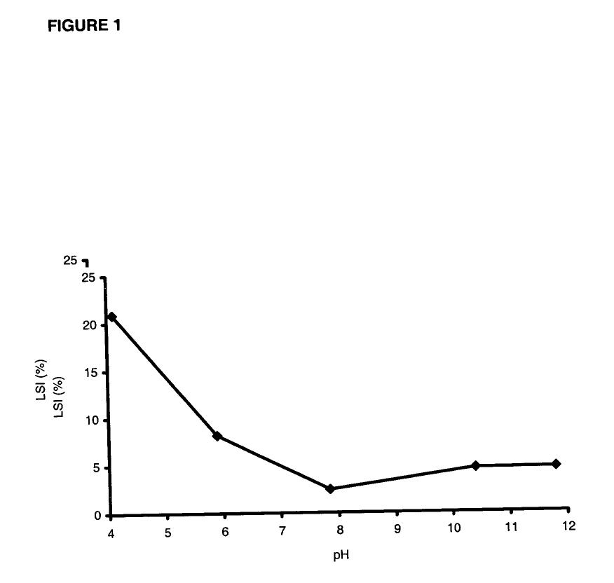

A stability study was conducted using slurries of crushed EKB-569 tablets (Table 3) in water at various pH conditions. Slurries were stored at 56° C. Stability results are summarized in FIG. 1. FIG. 1 shows that EKB-569 degradation in the slurry was more pronounced in the acidic region and most stable at pH values of 8 and higher. These findings were unexpected based on solution stability results discussed above.

Based on the above stability study, basic excipients were incorporated in the slurries to stabilize EKB-569. Basic excipients used included organic substances such as arginine and tromethamine, salts of organic substances such as EDTA tetra sodium, inorganic salts such as sodium carbonate, sodium bicarbonate, and calcium carbonate.

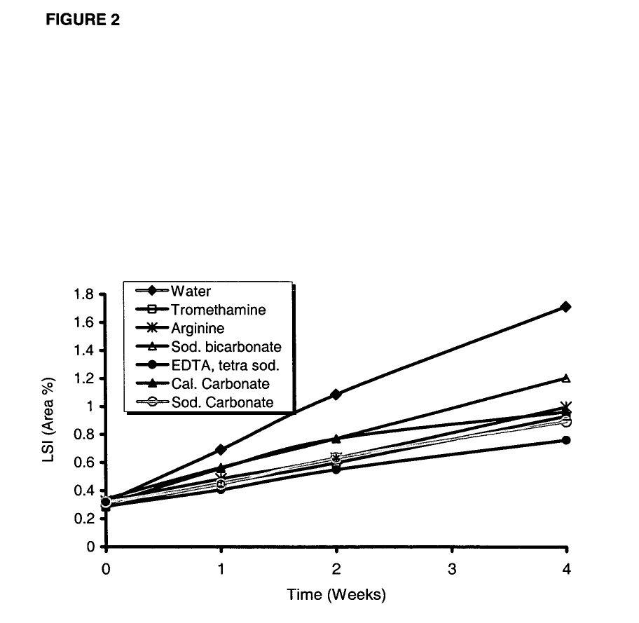

Slurries were prepared using 5% solutions or suspensions of basic excipients and crushed EKB-569 tablets. Reference slurry was prepared using crushed EKB-569 tablets and water. Stability of the slurries was studied at 56° C. FIG. 2 shows slurry stability results. All slurries exhibited improved stability as compared to the reference. Table 5 exhibits the pH of slurries containing EKB-569 granulation and excipients. The pH of the slurries was higher than 8 for all.

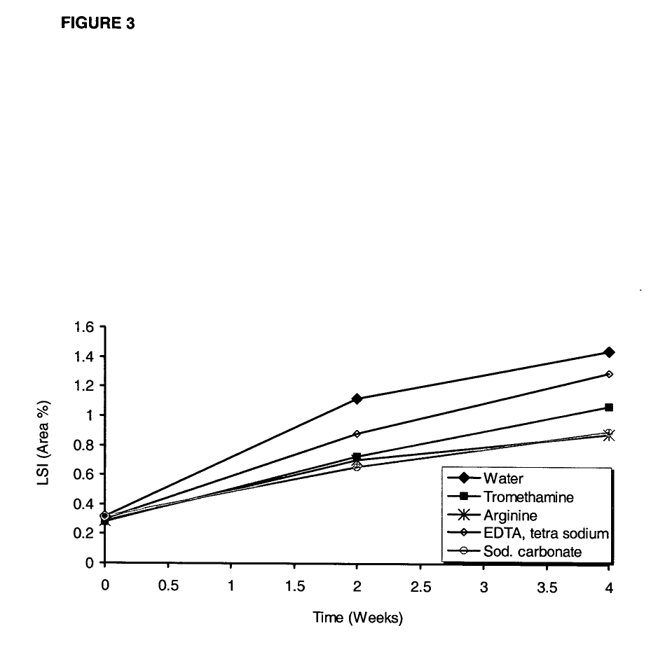

Slurries were also prepared using 1% solutions of tromethamine, arginine and sodium carbonate. FIG. 3 shows stability results for these slurries. Results indicate that all excipients were still able to decrease drug degradation from the slurry. The pH values for various slurries are listed in table 5. These values were found to be similar to that for corresponding slurries prepared using 5% excipient solutions. This further supported the discovery that pH values higher than 8 improves EKB-569 pharmaceutical composition stability.

TABLE 5
pH of excipient slurries prepared using crushed EKB-569 tablets and basic
excipients solution/suspension at various excipient concentrations.

| | pH at | | pH at | | pH at | | pH at | |
|---|---|---|---|---|---|---|---|---|
| | 5% excipient | | 1% excipient | | 0.5% excipient | | 0.1% excipient | |
| | Excipient | Tablet | Excipient | Tablet | Excipient | Tablet | Excipient | Tablet |
| | Solution | slurry | Solution | Slurry | Solution | Slurry | Solution | Slurry |
| Reference | 6.23 | 7.85 | | | | | | |
| (Water) | | | | | | | | |
| Tromethamine | 10.87 | 9.75 | 10.51 | 9.43 | 9.99 | 8.98 | 9.98 | 8.5 |
| Arginine | 11.23 | 10.11 | 10.91 | 9.62 | 10.57 | 9.41 | 10.18 | 8.61 |
| Sodium | 11.5 | 10.91 | 11.31 | 10.59 | 11.21 | 10.43 | 10.99 | 9.8 |
| carbonate | | | | | | | | |
| Sodium | 8.18 | 8.57 | 8.34 | 8.7 | 8.56 | 8.68 | 8.69 | 8.57 |
| bicarbonate | | | | | | | | |
| Calcium | 9.12 | 9.24 | 9.02 | 9.14 | 8.93 | 9.09 | 8.95 | 8.9 |
| carbonate | | | | | | | | |
| EDTA, tetra | 10.75 | 10.52 | 10.94 | 10.07 | 10.84 | 9.56 | 10.55 | 8.26 |
| sodium | | | | | | | | |

Based on the above results, basic excipients (arginine, tromethamine, calcium carbonate and sodium carbonate) were incorporated into EKB-569 tablet pharmaceutical composition at 0.1%, 0.5% and 1% levels. Table 6 shows the different tablet pharmaceutical compositions containing 1% basic excipients. Compositions of tablets containing 0.5% and 0.1% of these excipients remain the same. Quantity of microcrystalline cellulose was adjusted to allow for the changes in the amounts of basic excipients. Tablets were manufactured using granulation.

TABLE 6
Composition of various EKB-569 tablet pharmaceutical
compositions containing basic excipients^{a}.

| | Formula | Formula | Formula | Formula^{b} | Formula^{c} | Formula^{d} |
|---|---|---|---|---|---|---|
| Ingredients | 2 | 3 | 4 | 5 | 6 | 7 |
| EKB-569 | 7.143 | 7.143 | 7.143 | 7.143 | 7.143 | 7.143 |
| Microcrystalline cellulose | 46.357 | 46.357 | 46.357 | 46.357 | 46.357 | 46.257 |
| Lactose, Anhydrous | 40.0 | 40.0 | 40.0 | 40.0 | 40.0 | 40.0 |
| Sodium starch glycolate | 5.0 | 5.0 | 5.0 | 5.0 | 5.0 | 5.0 |
| Magnesium stearate | 0.5 | 0.5 | 0.5 | 0.5 | 0.5 | 0.5 |
| Sodium carbonate | 1.0 | — | — | — | — | — |
| Calcium carbonate | — | — | — | — | 1.0 | — |
| Tromethamine | — | 1.0 | — | — | — | 1.0 |
| Arginine | — | — | 1.0 | — | — | — |
| EDTA, tetra sodium | — | — | — | 1.0 | — | 0.1 |

^{a}Above table shows compositions of tablets containing 1% by weight of basic excipients. Compositions of tablets containing 0.5% and 0.1% of these excipients remains the same. Quantity of microcrystalline cellulose was adjusted to allow for the changes in the amounts of basic excipients.
^{b}EDTA, tetra sodium was used to manufacture tablets at the 1% level only.
^{c}Calcium carbonate was used at 1.0%, 0.5% and 0.1% level.
^{d}This pharmaceutical composition was prepared using a combination of basic excipients and was prepared by dry granulation process (roller compaction).

Tablets were stored at 40° C./75% RH for 1 month. Stability results for these pharmaceutical compositions are shown in Table 7. Results indicated that tablets containing various levels of the above basic excipients were more stable than the reference pharmaceutical composition.

TABLE 7
Level of total impurities (TI) for EKB-569 in
EKB-569 10 mg tablets containing basic
excipients at 0.1%, 0.5% and 1% levels. Samples
were stored in High Density Poly-ethylene (HDPE)
bottles at 40° C./75% RH for 1 month.

| | Total impurities TI % | | |
|---|---|---|---|
| | 0.1% basic | 0.5% basic | 1.0% basic |
| Basic excipient | excipient | excipient | excipient |
| Reference* | 4.72 | — | — |
| Arginine | 2.74 | 2.36 | 3.0 |
| Tromethamine | 2.87 | 2.35 | 2.24 |
| Sodium | 2.35 | 1.91 | 1.92 |
| carbonate | | | |
| Calcium | 3.04 | 2.91 | 2.37 |
| carbonate | | | |
| EDTA | 3 | — | — |

*Reference formulation in Table 3 (contains no basic excipients).

Another EKB-569 tablet pharmaceutical composition was prepared using 0.1% EDTA and 1% tromethamine. One month stability results at 40° C./75% RH showed that this pharmaceutical composition has total impurities of 2.37%. This is much less than the result obtained for the reference pharmaceutical composition (4.72%) under the same conditions.

TABLE 8
Composition of EKB-569 25 mg capsules
(stability results are shown in the FIG. below)

Pharmaceutical composition

| | Pharmaceutical | Pharmaceutical |
|---|---|---|
| | composition A | composition B |
| Ingredients | (% w/w) | (% w/w) |
| EKB-569 | 25 | 25 |
| Microcrystalline cellulose | 35 | 68 |
| Lactose | 34.5 | — |
| Sodium carbonate | — | 1 |
| Sodium starch glycolate | 5 | 5 |
| Magnesium stearate | 0.5 | 1.0 |

*Please note that earlier studies showed that lactose has no effect on stability of EKB-569 pharmaceutical composition

These experiments indicated that the presence of one or more basic excipients (arginine, tromethamine, calcium carbonate and sodium carbonate, and EDTA) in EKB-569 tablet pharmaceutical composition resulted in the improvement of its stability.

The preparation of the compounds of this invention encompassed by Formula 10 and Formula 11 are described below in Flowsheet B where Y, p, n, and m are as described above. X is selected from the group consisting of cycloalkyl or phenyl optionally substituted with one or more substituents selected from the group consisting of hydrogen, halogeno, alkyl of 1-6 carbon atoms, alkenyl of 2-6 carbon atoms, alkynyl of 2-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino. The moieties (R₁₀)ₖ represent 1 to 3 substituents on the aromatic ring that can be the same or different and are selected independently from the group hydrogen, halogeno, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, halomethyl, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, trifluoromethyl, cyano, nitro, carboxy, carboalkyl of 2-7 carbon atoms, phenoxy, phenyl, thiophenoxy, benzyl, alkoxyamino of 1-4 carbon atoms, dialkylamino of 2 to 12 carbon atom, N,N-dialkylaminoalkyl of 3-14 carbon atoms, phenylamino, benzylamino, N-alkylcarbamoyl of 1-6 carbon atoms, N,N-dialkylcarbamoyl of 2-12 carbon atoms. R₁₁ is a radical and is selected from the group:

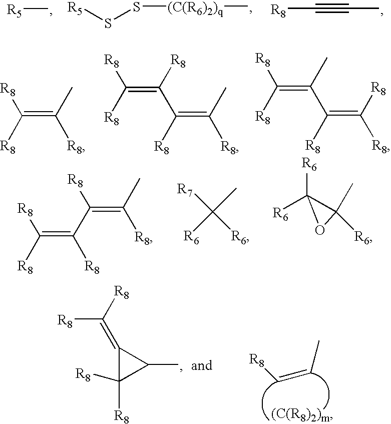

wherein q, m, R₅, R₆, R₇ , and R₈ are as defined above. According to the sequence of reactions outlined in Flowsheet B, acylation of 6 with either an acid chloride of Formula 8 or a mixed anhydride of Formula 9 (which is prepared from the corresponding carboxylic acid) in an inert solvent such as tetrahydrofuran (THF) in the presence of an organic base such as pyridine, triethylamine, or N-methyl morpholine gives the compounds of this invention represented by Formula 11. In those cases where 8 or 9 have an asymmetric carbon atom, they can be used as the racemate or as the individual R or S entantiomers in which case the compounds of this invention will be in the racemic or R and S optically active forms, respectively. Acylation of 6 with a cyclic anhydride of Formula 7 in an inert solvent such as tetrahydrofuran in the presence of a basic catalyst such as pyridine or triethylamine gives the compounds of the invention of Formula 10. The compounds of Formula 6 with p=0 can be prepared from the aromatic nitro substituted compounds by reducing the nitro group with a reducing agent such as iron and ammonium chloride in alcohol, sodium hydrosulfite in an aqueous mixture, or the like.

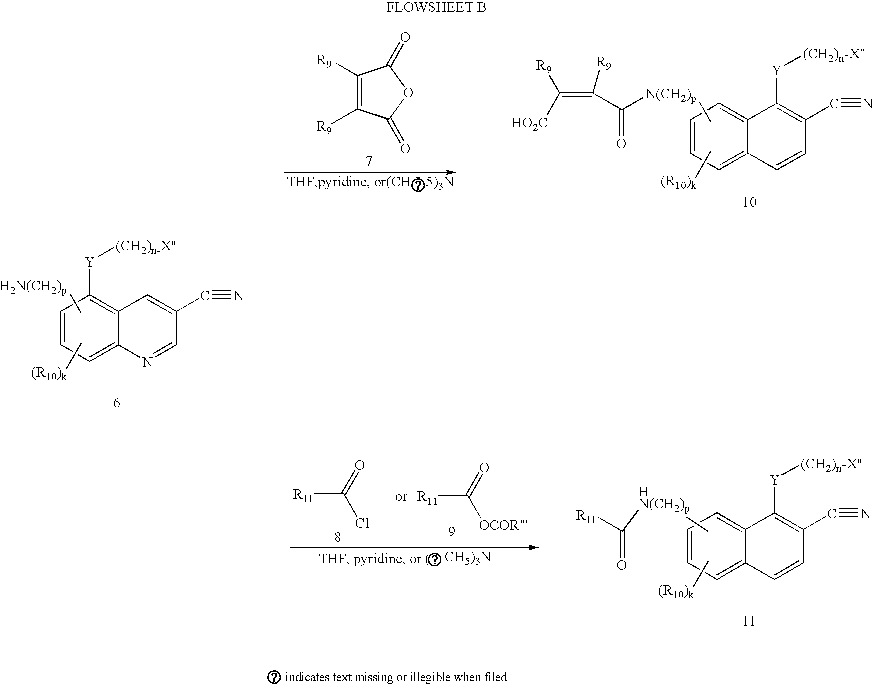

The following are representative examples of the compounds of this invention whose preparation is described in U.S. Pat. No. 6,002,008, or below:

### EXAMPLE 1

### 1,4-Dihydro-7-methoxy-4-oxo-3-quinolinecarbonitrile

### EXAMPLE 2

### 1,4-Dihydro-7-methoxy-6-nitro-4-oxo-3-quinolinecarbonitrile

### EXAMPLE 3

### 4-Chloro-7-methoxy-6-nitro -3-quinolinecarbonitrile

### EXAMPLE 4

### 4-[(3-Bromophenyl)amino]-7-methoxy-6-nitro -3-quinoline-carbonitrile

### EXAMPLE 5

### 6-Amino-4-[(3-bromophenyl)amino]-7-methoxy -3-quinoline carbonitrile

### EXAMPLE 6

### 2-Cyano-3-(4-nitrophenylamino)acrylic Acid Ethyl Ester

### EXAMPLE 7

### 1,4-Dihydroquinoline-6-Nitro-4-oxo -3-carbonitrile

### EXAMPLE 8

### 4-Chloro-6-nitro-3-quinolinecarbonitrile

### EXAMPLE 9

### 4-[(3-Bromophenyl)amino]-6-nitro-3-quinolinecarbonitrile

### EXAMPLE 10

### 6-Amino-4-[(3-bromophenyl)amino]-3-quinolinecarbonitrile

### EXAMPLE 11

### N-[4-[(3-Bromophenyl)amino]-3-cyano-6-quinolinyl]-2-propenamide

### EXAMPLE 12

### 3-Carbethoxy-4-hydroxy-6,7-dimethoxyquinoline

### EXAMPLE 13

### 4-Bromo-but-2-enoic acid [4-(3-bromo-phenylamino)-3-cyano-quinolin-6-yl]-amide

### EXAMPLE 14

### 4-Dimethylamino-but-2-enoic acid [4-(3-bromo-phenylamino)-3-cyano-quinolin-6-yl]-amide

### EXAMPLE 15

### 4-Diethylamino-but-2-enoic acid [4-(3-bromo-phenylamino)-3-cyano-quinolin-6-yl]-amide

### EXAMPLE 16

### 4-Methylamino-but-2-enoic acid [4-(3-bromo-phenylamino)-3-cyano-quinolin-6-yl]-amide

### EXAMPLE 17

### 4-Dimethylamino-but-2-enoic acid [4-(3-bromo-phenyl-amino)-3-cyano-7-methoxy-quinolin-6-yl]-amide

### EXAMPLE 18

### 4-Diethylamino-but-2-enoic acid [4-(3-bromo-phenyl-amino)-3-cyano-7-methoxy-quinolin-6-yl]-amide

### EXAMPLE 19

### 4-Morpholin-4-yl-but-2-enoic acid [4-(3-bromo-phenylamino)-3-cyano-7-methoxy-quinolin-6-yl]-amide

### EXAMPLE 20

### 4-(3-Chloro-4-fluoro-phenylamino)-7-methoxy-6-nitro-quinoline-3-carbonitrile

### EXAMPLE 21

### 6-Amino-4-(3-chloro-4-fluoro-phenylamino)-7-methoxy-quinoline-3-carbonitrile

### EXAMPLE 22

### 4-Dimethylamino-but-2-enoic acid [4-(3-chloro-4-fluoro-phenylamino)-3-cyano-7-methoxy-quinolin-6-yl]-amide

### EXAMPLE 23

### 4-Diethylamino-but-2-enoic acid [4-(3-chloro-4-fluoro-phenylamino)-3-cyano-7-methoxy-quinolin-6-yl]-amide

### EXAMPLE 24

### 4-Morpholin-4-yl-but-2-enoic acid [4-(3-chloro-4-fluoro-phenylamino)-3-cyano-7-methoxy-quinolin-6-yl]-amide

### EXAMPLE 25

### 4-(3-Bromo-4-fluoro-phenylamino)-7-methoxy-6-nitro-quinoline-3-carbonitrile

### EXAMPLE 26

### 6-Amino-4-(3-bromo-4-fluoro-phenylamino)-7-methoxy-quinoline-3-carbonitrile

### EXAMPLE 27

### 4-Dimethylamino-but-2-enoic acid [4-(3-bromo-4-fluoro-phenylamino)-3-cyano-7-methoxy-quinolin-6-yl]-amide

### EXAMPLE 28

### 4-Diethylamino-but-2-enoic acid [4-(3-bromo-4-fluoro-phenylamino)-3-cyano-7-methoxy-quinolin-6-yl]-amide

### EXAMPLE 29

### 7-Ethoxy-4-hydroxy-quinoline-3-carbonitrile

### EXAMPLE 30

### 7-Ethoxy-4-hydroxy-6-nitro-quinoline-3-carbonitrile

### EXAMPLE 31

### 4-Chloro-7-ethoxy-6-nitro-quinoline-3-carbonitrile

### EXAMPLE 32

### 4-(3-Bromo-phenylamino)-7-ethoxy-6-nitro-quinoline-3-carbonitrile

### EXAMPLE 33

### 6-Amino-4-(3-bromo-phenylamino)-7-ethoxy-quinoline-3-carbonitrile

### EXAMPLE 34

### 4-Bromo-but-2-enoic acid [4-(3-bromo-phenylamino)-3-cyano-7-ethoxy-quinolin-6-yl]-amide

### EXAMPLE 35

### 4-Dimethylamino-but-2-enoic acid [4-(3-bromo-phenyl-amino)-3-cyano-7-ethoxy-quinolin-6-yl]-amide

### EXAMPLE 36

### 4-Diethylamino-but-2-enoic acid [4-(3-bromo-phenylamino)-3-cyano-7-ethoxy-quinolin-6-yl]-amide

### EXAMPLE 37

### 4-Morpholin-4-yl-but-2-enoic acid [4-(3-bromo-phenylamino)-3-cyano-7-ethoxy-quinolin-6-yl]-amide

### EXAMPLE 38

### 8-Methoxy-4-hydroxy-6-nitro-quinoline-3-carbonitrile

### EXAMPLE 39

### 4-Chloro-8-methoxy-6-nitro-quinoline-3-carbonitrile

### EXAMPLE 40

### 6-nitro-4-(3-bromo-phenylamino)-8-methoxy-quinoline-3-carbonitrile

### EXAMPLE 41

### 6-Amino-4-(3-bromo-phenylamino)-8-methoxy-quinoline-3-carbonitrile

### EXAMPLE 42

### 4-Bromo-but-2-enoic acid [4-(3-bromo-phenylamino)-3-cyano-8-methoxy-quinolin-6-yl]-amide

### EXAMPLE 43

### 4-Dimethylamino-but-2-enoic acid [4-(3-bromo-phenyl-amino)-3-cyano-8-methoxy-quinolin-6-yl]-amide

### EXAMPLE 44

### 4-Diethylamino-but-2-enoic acid [4-(3-bromo-phenyl-amino)-3-cyano-8-methoxy-quinolin-6-yl]-amide

### EXAMPLE 45

### 4-Morpholin-4-yl-but-2-enoic acid [4-(3-bromo-phenyl-amino)-3-cyano-8-methoxy-quinolin-6-yl]-amide

### EXAMPLE 46

### 4-Dimethylamino-but-2-ynoic acid [4-(3-bromo-phenyl-amino)-3-cyano-7-methoxy-quinol-6-yl]-amide

### EXAMPLE 47

### (E)-But-2-enoic Acid [4-(3-Bromophenylamino)-3-cyanoquinolin-6-yl]amide

and

### EXAMPLE 48

### 4-Dimethylamino-but-2-enoic acid [4-(3-chloro-4-fluoro-phenylamino)-3-cyano-7-ethoxy-quinolin-6-yl]-amide

### 5-Methoxy-2-methyl-4-nitroacetanilide

A solution of 182.1 g (1.0 mol) of 5-methoxy-2-methyl-4-nitroaniline in 400 ml acetic acid was heated to reflux. To the hot solution was added 320 ml of acetic anhydride. The mixture was refluxed for 0.5 hour and then poured onto ice. The solid was collected and washed twice with water and once with concentrated NH₄OH (this step converts any di-acetate to mono-acetate). The solid is then air dried. The solid is dissolved in 1400 ml of boiling chloroform, treated with MgSO₄ and Norite, and filtered while hot. The filtrate was boiled and 500 ml of hexanes were added. The mixture was cooled in an ice bath. Solid was collected giving 145.9 g (65%) of the product as an orange solid.

### 5-Ethoxy-2-methyl-4-nitroacetanilide

A mixture of 186 g (830 mmol) of 5-methoxy-2-methyl-4-nitroacetanilide and 105.5 g (2.49 mol) of LiCl in 1115 ml of DMF was mechanically stirred at reflux for 12 hours without using a condenser. The dark orange solution was allow to cool to room temperature and then allowed to stand overnight. To the stirring solution was added 114.65 g (830 mmol) of powdered K₂CO₃ and 265.4 ml (3.32 mol) of ethyl iodide. The mixture was slowly heated with stirring. At about 70¼° C. a rapid gas evolution ensues. After most gas has evolved, heating is continued to reflux temperature. The mixture is refluxed for 5 hours and then poured onto ice water. The solid is collected, washed several times with water, and air dried. The solid is dissolved in 2 L of boiling chloroform, treated with MgSO₄, and filtered while hot. The filtrate is boiled and diluted with 1.5 L hexanes. The mixture is cooled and solid is collected giving 105 g of a yellow solid (53%).

### 2-Acetylamino-4-ethoxy-5-nitro-benzoic acid

A solution of 217.3 g of potassium permanganate and 75.23 g of magnesium sulfate in 5000 ml of water was heated to 80° C. Then 119 g (0.5 moles) of 5-ethoxy-2-methyl-4-nitroacetanilide was added in one portion. Heating at reflux was continued. After about 45 minutes (the disappearance of the permanganate color) an additional 37.62 g of magnesium sulfate and then 108.65 g of potassium permanganate were added. After about 45 minutes of additional reflux ( the disappearance of the permanganate color) the reaction was filtered hot. The manganese dioxide cake was reserved. Acidification of the filtrate with concentrated hydrochloric acid gave product. The reserved manganese dioxide was boiled with 2000 ml of water, and filtered. Acidification of the filtrate gave additional product. The products were combined and dried to give 68.19 g (50.8%) of the desired product. Starting material could be extracted from the manganese dioxide cake with acetone.

### 3-Ethoxy-4-nitroaniline

To 600 ml of H₂O was slowly added 400 ml concentrated H₂SO₄. To the hot mixture was added 118.5 g 0.44 mol)) of 2-acetylamino-4-ethoxy-5-nitro-benzoic acid. The mixture was heated to 110-112° C. with stirring. Initially there was a vigorous gas evolution. After 1 hour, the mixture was poured unto ice. The mixture was made basic with concentrated ammonium hydroxide (an exothermic reaction ensued). The mixture was allowed to cool to room temperature and the solid was collected by filtration. The solid was washed several time with 500 ml portions of water and then dried in vacuum and then extracted several times with warm ethyl acetate. The extracts were filtered and solvent was removed giving 57.8 g (71 %) of the product.

### 2-(2-Cyano-2-ethoxycarbonyl-vinylamino)-4-ethoxy-5-nitro-benzoic acid

A mixture of 58.96 g (0.324 moles) of 3-ethoxy -4-nitroaniline and 77.22 g (0.456 moles) of ethyl (ethoxymethylene) cyano acetate in 210 ml of toluene was refluxed for about 16 hours (overnight). The reaction was cooled in an ice bath, and the product was filtered. It was washed with three portions of ether, then dried to give 94.33 g (95.8%) of the desired product. This can be recrystallized in about 80% yield from methyl cellosolve.

### 7-Ethoxy-4-hydroxy-6-nitro-quinoline-3-carbonitrile

The yellow starting material 2-(2-cyano-2-ethoxycarbonyl-vinylamino)-4-ethoxy-5-nitro-benzoic acid (37.5 g, 0.123 mol), which had been recrystallized from 2-methoxyethanol, was added as a solid to 2.5 L of refluxing (256° C.) Dowtherm in a 5 L three-necked flask equipped with a mechanical stirrer and a thermometer under nitrogen. The reaction mixture was stirred vigorously at this temperature for 1.25 hours, and then allowed to cool to room temperature. The thick reaction mixture was diluted with 2L of ether, filtered and washed with ether to yield 24.2 g of the cyclized product 7-ethoxy-4-hydroxy-6-nitro-quinoline-3-carbonitrile as an off-white solid with a yield of 76%.

The filtrate was evaporated to remove ether and then treated with hexane. The resulting yellow precipitate was collected and washed with hexane to yield 10˜15% unreacted starting material, which could be recycled to generate more cyclized product. The resulting filtrate was evaporated to remove hexane and then passed through a thin pad of silica gel to remove colored impurities to regenerate the Dowtherm for more cyclization reactions.

### 4-Chloro-7-ethoxy-6-nitro-quinoline-3-carbonitrile

In a 1 L round-bottomed flask, the nitro compound 7-ethoxy-4-hydroxy-6-nitro-quinoline-3-carbonitrile (20 g, 77 mmol) was refluxed with 120 ml of phosphorus oxychloride under nitrogen for 2.5 hours. TLC (ethyl acetate: hexane=1:1) showed no starting material left. The volatile reagents were removed by rotary evaporation and further azeotropically removed with toluene at 50° C. The flask containing the solid residue was cooled in an ice bath, and 600 ml of methylene chloride was added to dissolve the residue. The resulting cold methylene chloride solution was added into a vigorously stirred solution of 250 ml ice-cold saturated potassium carbonate solution (53.3g, 5 eq) and stirred for 30 min. The organic layer was separated, washed and dried to give 18.58 g of 4-chloro-7-ethoxy-6-nitro-quinoline-3-carbonitrile with a yield of 86.9%.

### 4-(3-Chloro-4-fluoro-phenylamino)-7-ethoxy-6-nitro-quinoline-3-carbonitrile

4-Chloro-7-ethoxy-6-nitro-quinoline-3-carbonitrile (26.8 g 96.5 mmol) and 3-chloro-4-fluoroaniline 14.05 g 96.5 mmol) in 900 ml of iso-propanaol were refluxed under N₂ for 3.5 hours TLC (ethyl acetate: hexane=1:1) showed no starting material left. After standing at room temperature overnight, the hydrochloride salt was filtered off and washed with isopropanol and ether giving 4-(3-chloro-4-fluoro-phenylamino)-7-ethoxy-6-nitro-quinoline-3-carbonitrile 38.6 g (95%) as a yellow hydrochloride salt.

### 6-Amino-4-(3-chloro-4-fluoro-phenylamino)-7-ethoxy-quinoline-3-carbonitrile

4-(3-Chloro-4-fluoro-phenylamino)-7-ethoxy-6-nitro-quinoline-3-carbonitrile hydrochloride (38.6 g 91.2 mmol) was mixed with 35.7 g (638 mmol) of iron powder. A solution of 43.9 g (820 mmol) of ammonium chloride in 280 ml of water was added followed by 985 ml of methanol. The mixture was refluxed with mechanical stirring under nitrogen for 4 hour at which time TLC indicated complete reduction. The reaction mixture was filtered hot and solids were washed with 500 ml of boiling methanol. After the combined filtrate was evaporated, the residue was partitioned between 1.5 L of warm ethyl acetate and 700 ml of saturated sodium bicarbonate solution. The organic layer was dried over magnesium sulfate, treated with Norite, filtered and evaporated to give a solid which was recrystallized from CHCl₃— hexanes giving 29.0 g (89%) of 6-amino-4-(3-chloro-4-fluoro-phenylamino)-7-ethoxy-quinoline-3-carbonitrile as a light green solid.

### 4-Bromo-but-2-enoic acid [4-(3-chloro-4-fluoro-phenylamino)-3-cyano-quinolin-6-yl]-amide

To 14.98 g (63.17 mmol) of trimethylsilyl 4-bromo-2-butenoate (prep.:Synthesis 745 1983) in 36 ml of methylene chloride, was added 8.82 g (69.5 mmol) of oxalyl chloride, followed by 1 drop of dried DMF. After the solution was stirred for 2 hours, the solvent was evaporated, and further azeotropically distilled with carbon tetrachloride to yield the acid chloride.

6-Amino-4-(3-chloro-4-fluoro-phenylamino)-7-ethoxy-quinoline-3-carbonitrile (19.6 g, 54.9 mmol) was mixed with 11.46 ml (65.91 mmol) of N,N-diisopropylethylamine in 366 ml of anhydrous THF under nitrogen in an ice bath. A solution of the acid chloride prepared above in 183 ml of THF was added over 15 minutes, and then stirred for half an hour at 0° C. The reaction vessel was sealed and stored in the freezer overnight.

The reaction solution was rotary evaporated and the residue was partitioned between saturated sodium bicarbonate and ethyl acetate. The organic layer was separated, washed, dried with magnesium sulfate and passed through a thin layer of silica gel to give 32 g of the crude product as an orange solid. The crude product was refluxed with 400 ml of methanol for half an hour. After cooling to room temperature, the solid was collected and washed with methanol followed by hexane to give 21.3 g of beige solid with a yield of 76.5%. It is a mixture of the bromo and chloro compounds. More product could be isolated from the mother liquor.

### 4-Dimethylamino-but-2-enoic acid [4-(3-chloro-4-fluoro-phenylamino)-3-cyano-7-ethoxy-quinolin-6-yl]-amide

The bromo/chloro compounds (19.88 g, 39.53 mmol) were dissolved in 800 ml of THF at 0° C. and 2 equivalent of 2M dimethylamine (39.54 ml, 79.07 mmol) in THF was added in one portion. The reaction solution was stirred at room temperature overnight. Another equivalent of dimethylamine was added. After stirring overnight at room temperature, only 10% of chloro compound was unreacted.

The reaction solution was rotary evaporated and the residue was partitioned between ethyl acetate and saturated potassium bicarbonate. The organic layer was dried, filtered and evaporated to give 17 g of orange glass. The crude product was taken up in acetone and purified by column chromatography using acetone as the eluant. The main fractions were pooled and evaporated to give 9.8 g of a yellow glass. It was then dissolved in 350 ml of hot ethyl acetate and evaporated to a concentrated solution. A few drops of methanol was added to assist recrystallization. After standing at room temperature overnight, the beige crystals were filtered to yield 7.09 g of pure 4-dimethylamino-but-2-enoic acid [4-(3-chloro-4-fluoro-phenylamino)-3-cyano-7-ethoxy-quinolin-6-yl]-amide (mp 196-198° C.) with a yield of 38.7%. A lot of product remained in the mother liquors in the steps of chromatography and recrystallization, and could be isolated. The expected yield is about 60%.

Details of the preparation of the compounds of examples 1-47 are found in U.S. Pat. No. 6,002,008, issued Dec. 14, 1999 and the preparation steps are hereby incorporated by reference.

What is claimed is:

1. A stabilized pharmaceutical composition comprising a compound of the formula I: 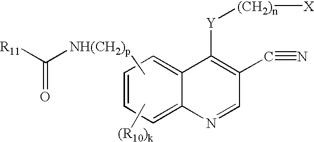
wherein:
X is selected from the group consisting of cycloalkyl or phenyl optionally substituted with one or more substituents selected from the group consisting of hydrogen, halogen, alkyl of 1-6 carbon atoms, alkenyl of 2-6 carbon atoms, alkynyl of 2-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino. The moieties (R₁₀)ₖ represent 1 to 3 substituents on the aromatic ring that can be the same or different and are selected independently from the group hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, halomethyl, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, trifluoromethyl, cyano, nitro, carboxy, carboalkyl of 2-7 carbon atoms, phenoxy, phenyl, thiophenoxy, benzyl, alkoxyamino of 1-4 carbon atoms, dialkylamino of 2 to 12 carbon atom, N,N-dialkylaminoalkyl of 3-14 carbon atoms, phenylamino, benzylamino, N-alkylcarbamoyl of 1-6 carbon atoms, N,N-dialkylcarbamoyl of 2-12 carbon atoms. R₁₁ is a radical and is selected from the group: 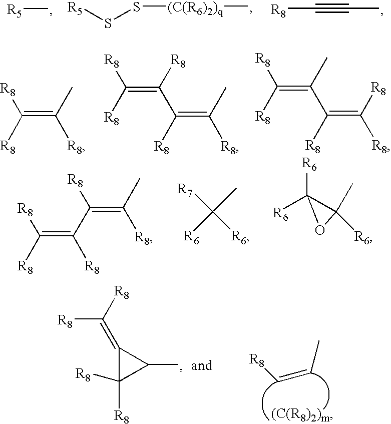
n is 0-1;
Y is —NH—, —O—, —S—, or —NR—;
R is alkyl of 1-6 carbon atoms;
R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₆ is hydrogen, alkyl of 1-6 carbon atoms, or alkenyl of 2-6 carbon atoms;
R₇ is chloro or bromo;
R₈ is hydrogen, alkyl of 1-6 carbon atoms, aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
k=1-3, q=1-3, m=1-3, and p=0-3; or a pharmaceutically acceptable salt thereof;
said pharmaceutical composition containing at least one basic excipient in a concentration sufficient to bring the pH of the composition to at least 8, and at least one pharmaceutically acceptable excipient.

2. The stabilized pharmaceutical composition of claim 1 wherein the basic excipient comprises sodium bicarbonate, ammonium carbonate, glycine, arginine, tromethamine, calcium carbonate, or sodium carbonate alone or in combination.

3. The stabilized pharmaceutical composition of claim 1 wherein the basic excipient comprises arginine, tromethamine, calcium carbonate or sodium carbonate alone or in combination.

4. The stabilized pharmaceutical composition of claim 1 wherein the pH of the composition is from about 8 to about 13.5.

5. The stabilized pharmaceutical composition of claim 1 wherein the pH of the composition is from about 8 to about 10.

6. The stabilized pharmaceutical composition of claim 1 wherein the pH of the composition is about 8.

7. The stabilized pharmaceutical composition of claim 1 wherein the basic excipient or combination of basic excipients comprises about 0.1% to about 50% by weight of the pharmaceutical composition.

8. The stabilized pharmaceutical composition of claim 1 wherein the basic excipient or combination of basic excipients comprises about 0.25% to about 10% by weight of the pharmaceutical composition.

9. The stabilized pharmaceutical composition of claim 1 wherein the basic excipient or combination of basic excipients comprises from about 0.5% to about 5% by weight of the pharmaceutical composition.

10. The stabilized pharmaceutical composition of claim 1 in the form of a solid dosage, a semi-solid, or suspension.

11. The stabilized pharmaceutical composition of claim 10 wherein the solid dosage form consists of a powder, a sphere, a capsule, or a tablet.

12. The stabilized pharmaceutical composition of claim 10 wherein the solid dosage, semi-solid, or suspension form comprises an immediate release form.

13. The stabilized pharmaceutical composition of claim 10 wherein the solid dosage, semi-solid, or suspension form comprises a sustained release form.

14. The stabilized pharmaceutical composition of claim 10 wherein the solid dosage form is enteric coated.

15. The stabilized pharmaceutical composition comprising a compound of the formula: 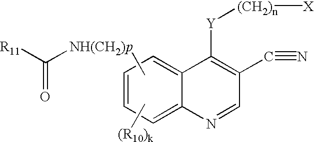
wherein:
X is a phenyl optioanlly substituted with a halogen;
n is 0-1;
Y is NH;
(R₁₀)ₖ is hydrogen, methoxy, ethoxy;
k=1-3, and p=0-3;
R₁₁ is 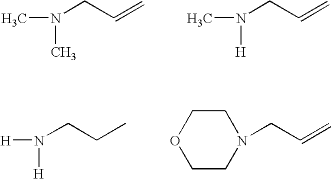
said pharmaceutical composition containing at least one basic excipient in a concentration sufficient to bring the pH of the composition to at least 8, and at least one pharmaceutically acceptable excipient.

16. The stabilized pharmaceutical composition of claim 1 wherein the compound comprises:
N-[4-[(3-bromophenyl)amino]-3-cyano-6-quinolinyl]-2-propenamide;
4-dimethylamino-but-2-enoic acid [4-(3-bromo-phenylamino)-3-cyano-quinolin-6-yl]amide;
4-methylamino-but-2-enoic acid [4-(3-bromo-phenylamino)-3-cyano-quinolin-6-yl]amide;
4-dimethylamino-but-2-enoic acid [4-(3-bromo-phenyl-amino)-3-cyano-7-ethoxy-quinolin-6-yl]amide;
4-morpholino-4-yl-but-2-enoic acid [4-(3-bromo-phenyl-amino)-3-cyano-7-ethoxy-quinolin-6-yl]amide;
4-morpholino-4-yl-but-2-enoic acid [4-(3-bromo-phenyl-amino)-3-cyano-8-methoxy-quinolin-6-yl]amide;
4-dimethylamino-but-2-enoic acid [4-(3-bromo-phenyl-amino)-3-cyano-7-methoxy-quinolin-6-yl]amide;
4-dimethylamino-but-2-enoic acid [4-(3-chloro-4-fluoro-phenyl-amino)-3-cyano-7-ethoxy-quinolin-6-yl]amide;
4-dimethylamino-but-2-enoic acid [4-(3-bromo-phenyl-amino)-3-cyano-7-methoxy-quinolin-6-yl]amide; or
4-morpholino-4-yl-but-2-enoic acid [4-(3-bromo-phenyl-amino)-3-cyano-7-methoxy-quinolin-6-yl]amide.

17. The stabilized pharmaceutical composition of claim 1 wherein the compound comprises 4-dimethylamino-but-2-enoic acid [4-(3-chloro-4-fluoro-phenylamino)-3-cyano-7-ethoxy-quinolin-6-yl]-amide.

18. A method of stabilizing a compound of the formula: 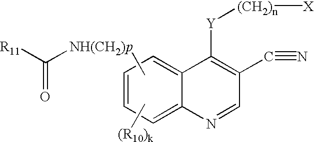
wherein:
X is selected from the group consisting of cycloalkyl or phenyl optionally substituted with one or more substituents selected from the group consisting of hydrogen, halogeno, alkyl of 1-6 carbon atoms, alkenyl of 2-6 carbon atoms, alkynyl of 2-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino. Each R₉ is independently hydrogen, phenyl, or alkyl of 1-6 carbon atoms. The moieties (R₁₀)ₖ represent 1 to 3 substituents on the aromatic ring that can be the same or different and are selected independently from the group hydrogen, halogeno, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, halomethyl, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, trifluoromethyl, cyano, nitro, carboxy, carboalkyl of 2-7 carbon atoms, phenoxy, phenyl, thiophenoxy, benzyl, alkoxyamino of 1-4 carbon atoms, dialkylamino of 2 to 12 carbon atom, N,N-dialkylaminoalkyl of 3-14 carbon atoms, phenylamino, benzylamino, N-alkylcarbamoyl of 1-6 carbon atoms, N,N-dialkylcarbamoyl of 2-12 carbon atoms. R₁₁ is a radical and is selected from the group: 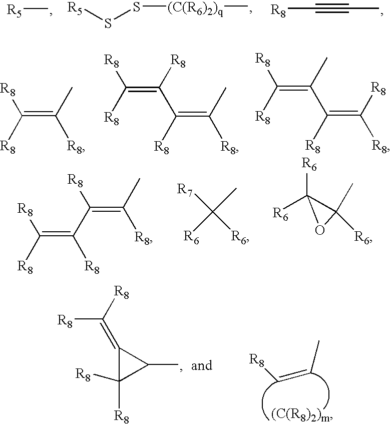
n is 0-1;
Y is —NH—, —O—, —S—, or —NR—;
R is alkyl of 1-6 carbon atoms;
R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₆ is hydrogen, alkyl of 1-6 carbon atoms, or alkenyl of 2-6 carbon atoms;
R₇ is chloro or bromo;
R₈ is hydrogen, alkyl of 1-6 carbon atoms, aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
k=1-3, q=1-3, m=1-3, and p=0-3; or a pharmaceutically acceptable salt thereof;
which comprises dry blending, dry granulating or wet granulating said compound with one or more pharmaceutically acceptable excipients and one or more basic excipients to form a pharmaceutical composition, said basic excipient(s) being in an amount sufficient to bring the pH of the composition to at least 8.

19. The method of claim 18 wherein the basic excipient comprises sodium bicarbonate, ammonium carbonate, glycine, arginine, tromethamine, calcium carbonate or sodium carbonate alone or in combination.

20. The method of claim 18 wherein the basic excipient comprises arginine, tromethamine, calcium carbonate or sodium carbonate alone or in combination.

21. The method of claim 18 wherein the pH of the composition is from about 8 to about 13.5.

22. The method of claim 18 wherein the pH of the composition is from about 8 to about 10.

23. The method of claim 18 wherein the pH of the composition is about 8.

24. The method of claim 18 wherein the basic excipient(s) and the combination comprises about 0.1% to about 50% by weight of the pharmaceutical composition.

25. The method of claim 18 wherein the basic excipient(s) comprises about 0.25% to about 10% by weight of the pharmaceutical composition.

26. The method of claim 18 wherein the basic excipient(s) comprises about 0.5% to about 5% by weight of the pharmaceutical composition.

27. The method of claim 18 wherein the pharmaceutical composition is in the form of a solid dosage, a semi-solid, or a suspension.

28. The method of claim 27 wherein the solid dosage form consists of a powder, a sphere, a capsule, or a tablet.

29. The method of claim 27 wherein the solid dosage, semi-solid, or suspension form comprises an immediate release form.

30. The method of claim 27, wherein the solid dosage, semi-solid, or suspension form comprises a sustained release form.

31. The method of claim 27 wherein the solid dosage form can be enteric coated.

32. The method of claim 18 comprising a compound of the formula: 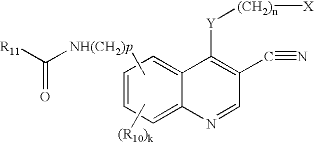
wherein:
X is a phenyl optionally substituted with a halogen;
n is 0-1; Y is NH;
(R₁₀)ₖ is hydrogen, methoxy, ethoxy; k=1-3, and p=0-3;
R₁₁ is 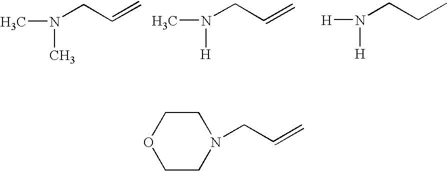
said pharmaceutical composition containing at least one basic excipient in a concentration sufficient to bring the pH of the composition to at least 8, and at least one pharmaceutically acceptable excipient.

33. The stabilized pharmaceutical composition of claim 18 wherein the compound comprises:
N-[4-[(3-bromophenyl)amino]-3-cyano-6-quinolinyl]-2-propenamide;
4-dimethylamino-but-2-enoic acid [4-(3-bromo-phenylamino)-3-cyano-quinolin-6-yl]amide;
4-methylamino-but-2-enoic acid [4-(3-bromo-phenylamino)-3-cyano-quinolin-6-yl]amide;
4-dimethylamino-but-2-enoic acid [4-(3-bromo-phenyl-amino)-3-cyano-7-ethoxy-quinolin-6-yl]amide;
4-morpholino-4-yl-but-2-enoic acid [4-(3-bromo-phenyl-amino)-3-cyano-7-ethoxy-quinolin-6-yl]amide;
4-morpholino-4-yl-but-2-enoic acid [4-(3-bromo-phenyl-amino)-3-cyano-8-methoxy-quinolin-6-yl]amide;
4-dimethylamino-but-2-enoic acid [4-(3-bromo-phenyl-amino)-3-cyano-7-methoxy-quinolin-6-yl]amide;
4-dimethylamino-but-2-enoic acid [4-(3-chloro-4-fluoro-phenyl-amino)-3-cyano-7-ethoxy-quinolin-6-yl]amide;
4-dimethylamino-but-2-enoic acid [4-(3-bromo-phenyl-amino)-3-cyano-7-methoxy-quinolin-6-yl]amide; or
4-morpholino-4-yl-but-2-enoic acid [4-(3-bromo-phenyl-amino)-3-cyano-7-methoxy-quinolin-6-yl]amide.

34. The method of claim 18 wherein the compound comprises 4-dimethylamino-but-2-enoic acid [4-(3-chloro-4-fluoro-phenylamino)-3-cyano-7-ethoxy-quinolin-6-yl]-amide.
